(19) United States
(12) Patent Application Publication
(10) Pub. No.: US 2022/0195541 A1
(43) Pub. Date: Jun. 23, 2022

(54) DETECTING A TARGET NUCLEIC ACID IN A BIOLOGICAL SAMPLE

(71) Applicant: PerkinElmer Health Sciences, Inc., Waltham, MA (US)

(72) Inventors: Yanhong Tong, Waltham, MA (US); Macy Veling, Waltham, MA (US); Eleanore Dougherty, Waltham, MA (US)

(21) Appl. No.: 17/556,209

(22) Filed: Dec. 20, 2021

Related U.S. Application Data
(60) Provisional application No. 63/129,209, filed on Dec. 22, 2020.

(51) Int. Cl. C12Q 1/70; C12Q 1/6806

(52) U.S. Cl. CPC C12Q 1/701; C12Q 1/6806

(57) ABSTRACT

Provided herein are methods, compositions, and kits for detecting a target nucleic acid, such as from a virus, in a biological sample. More specifically, the methods, compositions, and kits described herein describe detection of target nucleic acid from a coronavirus, such as SARS-CoV-2 coronavirus, with non-ionic detergents and isothermal amplification.

### CROSS-REFERENCE TO RELATED APPLICATIONS

This application claims priority to U.S. Provisional Patent Application No. 63/129,209, filed on Dec. 22, 2020, the entire contents of which are hereby incorporated by reference.

### BACKGROUND

This document describes technology for detecting the presence of a virus in a biological sample, including by the use of PCR based methods.

Viruses, including DNA viruses and RNA viruses, which can infect humans are generally not thermostable when heated. For example, the RNA virus SARS-CoV-2 is not stable at 56° C. for more than 30 min (Chin, A., et al., Stability of SARS-CoV-2 in different environmental conditions, The Lancet, 1(1), E 10, doi: doi.org/10.1016/S2666-5247(20)30003-3 (2020)). Additionally, the World Health Organization (WHO) has also reported that heating the SARS CoV (SARS coronavirus) at 56° C. for 15 minutes resulted in a loss in infectivity. However, heating biological samples at 56° C. or higher is not a desired temperature to enable reverse transcription for most commercially available reverse transcriptases or RNase inhibitors due to heat sensitivity.

Many viruses are enveloped in a lipid bilayer. The presence of the lipid bilayer makes nearly all enveloped viruses (e.g., RNA or DNA viruses) vulnerable to rapid inactivation by organic solvents (e.g., alcohol), detergents, and heat. For example, treating viruses with a mixture of solvent and detergent (SDS) has successfully been applied to inactivate most viruses of the transfusion relevant class of viruses without affecting the therapeutic properties of associated products (Rabenau, H.F., et al., SARS-coronavirus (SARS-CoV) and the safety of a solvent/detergent (S/D) treated immunoglobulin preparation, Biologicals, 33(2): 95-9, doi: 10.1016/j.biologicals.2005.01.003 (2005)). Furthermore, solvents such as tri-n-butyl phosphate (TnBP) and detergents such as Triton® X-100 and Tween® 80 are commonly used for viral inactivation (Solvent-Detergent Viral Inactivation of Plasma-Derived Products in Mobius® Single-Use Process Containers, Application Note, EMD Millipore (2015)). However, problematically, some products include chemicals that inhibit nucleic acid amplification and/or detection.

Isothermal amplification and polymerase chain reaction (PCR) have been widely applied for molecular diagnostics. Quantitative polymerase chain reaction (qPCR), also referred to as real-time PCR, is a method by which the amount of the PCR product can be determined in real-time using a fluorescent reporter. Generally, there are two qPCR detection methods. First, a qPCR detection method can be based on sequence-specific probes. One example of such detection method are TaqMan probes used in a TaqMan assay. Second, a qPCR detection method can be based on generic non-sequence specific double-stranded DNA (dsDNA) binding dyes. One example of such a dye is SYBR® Green. Generally, the TaqMan assay is the preferred qPCR method used in molecular diagnostics since the assay allows for sequence specific (e.g., gene specific) detection.

Nucleic acid extraction is typically required for molecular diagnostics of infectious diseases, including viral infections, such as SARS-CoV-2, and nucleic acids are usually extracted from a biological sample separately prior to qPCR amplification and detection. As such, nucleic acid extraction is time-consuming in molecular diagnostic assays.

### SUMMARY

The present disclosure generally describes compositions, methods, and kits for direct real-time PCR (e.g., quantitative PCR) target nucleic acid (e.g., viral) detection in biological samples without an additional target nucleic extraction procedure or subjecting the biological sample (or the target/viral nucleic acid) to a lysis. Non-ionic detergents allow amplification and detection to occur in a single solution in a single sample container without the need for lysis or a prior nucleic acid extraction. Described herein are methods, compositions, and kits for amplifying and detecting a target nucleic acid, e.g., from a virus (e.g., an RNA virus) in a biological sample in a single solution or mixture in a single container, where a combination of the biological sample and the single solution is created prior to subjecting the target or viral nucleic to a nucleic acid extraction or a lysis.

Provided herein are methods for detecting a presence of a target nucleic acid in a biological sample, the method including: creating a mixture in a container, the mixture including: the biological sample, a non-ionic detergent, one or more primers for specifically binding to the target nucleic acid in the biological sample, or a complement thereof, one or more probes for the target nucleic acid, and one or more polymerases, where the mixture is created prior to subjecting the target nucleic acid to a nucleic acid extraction or lysis; incubating the mixture to react the non-ionic detergent with the biological sample; amplifying the target nucleic acid, or a complement thereof, by polymerization using the one or more polymerases to generate an amplified target nucleic acid product, or a complement thereof and detecting the amplified target nucleic acid product or complement thereof with the one or more probes, thereby detecting the presence of the target nucleic acid in the biological sample.

In some embodiments, the non-ionic detergent is selected from a group consisting of: Tween 20, Tween 80, Triton X-100, NP 40, ECOSURF™ SA, Brij-58, and combinations thereof.

In some embodiments, the non-ionic detergent is present at a concentration from about 0.01% to about 10.0%.

In some embodiments, the biological sample is lysed for a period of time from about 30 seconds to about 20 minutes at a temperature from about 35° C. to about 75° C.

In some embodiments, the method includes contacting the mixture with an RNase inhibitor and/or a uracil-DNA glycosylase.

In some embodiments, the target nucleic acid is a viral nucleic acid including DNA. In some embodiments, the target nucleic acid is a viral nucleic acid including RNA.

In some embodiments, the viral nucleic acid is reverse transcribed using a reverse transcriptase selected from a group consisting of: MMLV, MMLV (RNase H minus), SuperScript II, SuperScript III, SuperScript IV, RevertAid H Minus, Maxima H, ProtoScript II, EnzScript™, ABscript II, EpiScript™, or RocketScript (Bioneer), and combinations thereof two or more times at a temperature of about 50° C. to about 70° C. In some embodiments, the viral nucleic acid includes viral nucleic acid from a bacteriophage, where the bacteriophage is an MS2 bacteriophage. In some embodiments, the method includes detecting a control nucleic acid, where the control nucleic acid is a MS2 bacteriophage gene.

In some embodiments, the viral nucleic acid includes viral nucleic acid from a coronavirus and, optionally, where the coronavirus includes a SARS-CoV-2 virus. In some embodiments, the method includes detecting the SARS-CoV-2 virus in the biological sample, where detecting the SARS-CoV-2 virus in the biological sample includes detecting a SARS-CoV2 N gene and/or a SARS-CoV-2 ORF gene. In some embodiments, the biological sample is obtained from a human, and optionally, where diagnosing the human with COVID-19 disease includes detecting the SARS-CoV-2 virus in the human biological sample.

In some embodiments, the method includes isothermally amplifying the target nucleic acid, where isothermally amplifying includes one of a helicase-dependent amplification, a loop mediated isothermal amplification, a recombinase polymerase amplification, or a rolling circle amplification.

In some embodiments, the one or more polymerases includes a DNA polymerase.

In some embodiments, the amplified target nucleic acid product is detected using a quantitative PCR method with the one or more probes. In some embodiments, the quantitative PCR method includes a real-time PCR assay and, optionally, where the quantitative PCR method includes a TaqMan™ assay. In some embodiments, the quantitative PCR method employs a non-sequence-specific double-stranded DNA-binding dye to detect the amplified target nucleic acid product and where the non-sequence specific double-stranded DNA binding dye is SYBR green.

Also provided herein are methods for detecting the presence of a viral nucleic acid in a biological sample, the methods including incubating a non-ionic detergent with the biological sample to react the non-ionic detergent with the biological sample; prior to subjecting the viral nucleic acid to a nucleic acid extraction or lysis, contacting the biological sample and the non-ionic detergent with a mixture including: one or more primers for specifically binding to the viral nucleic acid in the biological sample, or a complement thereof, one or more probes for the viral nucleic acid, and one or more polymerases; amplifying the viral nucleic acid, or a complement thereof, by polymerization using the one or more polymerases to generate an amplified viral nucleic acid product, or a complement thereof; and detecting the amplified viral nucleic acid product, or complement thereof, with the one or more probes, thereby detecting the presence of the virus in the biological sample.

Also provided herein are kits including (i) one or more polymerases; (ii) one or more primers for a viral nucleic acid; (iii) a non-ionic detergent; (iv) one or more probes; and (v) instructions for creating a mixture including a biological sample containing the viral nucleic acid, the one or more polymerases, the one or primers, the non-ionic detergent, and the one or more probes, where the mixture is created prior to subjecting the viral nucleic acid to a nucleic acid extraction or a lysis.

### DESCRIPTION OF DRAWINGS

FIG. 1 is an exemplary scheme to detect a virus in a biological sample. The multi-steps (lysis, nucleic acid release, primer binding, and reverse transcription under the protection of RNase inhibitors) can happen in a same tube/vial/well.

FIG. 2 is a chart indicating the mean Ct threshold of the N, ORF1ab, and IC genes under various lysis conditions.

FIG. 3 is a graph showing a qPCR amplification plot detecting a virus under various conditions.

FIG. 4 is a graph showing a workflow of virus process and detection. The swabs with virus samples are processed/resuspended in a buffer without lysis components, and then the resuspended virus can be added directly to reagents for lysis, amplification, and detection in a same tube/vial/well.

### DETAILED DESCRIPTION

To facilitate understanding of the disclosure set forth herein, terms are defined below. Generally, the nomenclature used herein and the laboratory procedures in biochemistry, genetics, molecular biology, and molecular diagnostics described herein are those well-known and commonly employed in the art. Other features, objects, and advantages of the invention will be apparent from the description and drawings, and from the claims. Unless defined otherwise, all technical and scientific terms used herein generally have the same meaning as commonly understood by one of ordinary skill in the art to which this disclosure belongs. Methods and materials are described herein for use in the present invention; other, suitable methods and materials known in the art can also be used. The materials, methods, and examples are illustrative only and not intended to be limiting. Each of the patents, applications, published applications, and other publications that are mentioned throughout the specification and the attached appendices are incorporated herein by reference in their entireties. In case of conflict, the present specification, including definitions, will control.

The term “biological sample” refers to a sample from an animal, including, but not limited to, a primate (e.g., human), monkey, cow, pig, sheep, goat, horse, dog, cat, rabbit, rat, or mouse. In some embodiments, the biological sample is a human sample. The term “biological sample” also refers to a sample with a control nucleic acid. For example, a biological sample used as a control can include a viral nucleic acid from a different virus. In some embodiments, the control viral nucleic acid is from a bacteriophage. In some embodiments, the bacteriophage is an MS2 bacteriophage. In some embodiments a biological sample includes a target nucleic acid. In some embodiments, a biological sample includes a control nucleic acid. It can thus be understood that a biological sample comprising a nucleic acid as provided herein may include: a blood sample, such as, for example, a liquid whole blood sample, components of whole blood, such as, but not limited to, red blood cells, white blood cells, or plasma, or components of whole blood or whole blood in combination with other substances; urine; saliva; vaginal or seminal fluids; fecal matter; excretions or secretions from the human body; nasopharyngeal samples; oropharyngeal samples; and, other samples comprising a target nucleic acid(s).

The term “non-ionic detergent” refers to detergents characterized by uncharged, hydrophilic headgroups. Generally, non-ionic detergents are based on polyoxyethylene such as for example, Tween®, Triton, NP-40, ECOSURF™ SA and Brij detergents, or a glycoside such as for example octyl thioglucoside and maltosides.

The term “target nucleic acid” refers to a nucleic acid to be detected by the methods and kits described herein, including, but not limited to a viral nucleic acid, a bacterial nucleic acid, an intracellular or extracellular nucleic acid, an endogenous nucleic acid, or an exogenous nucleic acid in a biological sample.

The term “lysis” as used herein refers to a process of breaking apart larger particles into smaller particles, such as to break down molecularly into smaller molecules, and can occur both cellularly (to the cell), inside a cell (intracellularly) and outside (extracellularly) of a cell. Lysis can thus refer to cellular, intracellular, and/or extracellular components of a biological sample, and can include, e.g., rupture of a cell membrane, rupture of a virus envelope, the breaking apart of a virus into smaller molecules such as DNA, RNA, lipids and proteins, etc. Accordingly, as used here, references to lysis of a biological sample includes but is not limited to the breaking down of the biological sample's cellular and non-cellular components, intracellularly and extracellularly, e.g., cells are broken down into subcellular components and further into molecular components, a virus is broken down into smaller molecules, etc. Lysis may be performed using heat, light, chemicals, ultrasound, mechanical, or other implements, and the disclosed methods and systems shall not be limited to the lysis technique.

In some embodiments, the target nucleic acid is viral nucleic acid. For example, the viral nucleic acid can be present in a biological sample derived from animal as described above. In some embodiments, the viral nucleic acid is DNA (e.g., a DNA virus). In some embodiments, the viral nucleic acid is RNA (e.g., a RNA virus). In some embodiments, the RNA virus is a coronavirus. In some embodiments, the coronavirus is the SARS-CoV-2 virus. In some embodiments, the target nucleic acid is an endogenous nucleic acid.

Biological sample heat lysis, for example, heating the biological sample at about 70° C. to about 95° C. for about 5 to about 10 minutes, is a typical solution to avoid separate nucleic acid extraction steps. However, biological sample heat lysis typically requires additional steps during PCR amplification which increases the overall sample processing time. The additional steps can include the addition of reverse transcriptase after heat lysis, since reverse transcriptase is typically heat sensitive and can be inactivated at such high temperatures. Generally, RNase inhibitors are also heat sensitive and are required to be added after the biological sample heat lysis. These additional steps are time consuming. One such example of an assay requiring heat lysis is the Lyra Direct SARS-CoV-2 Assay (Quidel, San Diego, Calif., USA). The Lyra Direct SARS-CoV-2 assay requires a biological sample heat lysis at 95° C. for 10 minutes, followed by a separate addition of the RT-PCR reagents. In some embodiments, methods disclosed herein do not require biological sample heat lysis, for example, all reagents for the detection of a target nucleic acid (e.g., from a virus) in a biological sample are in a single solution or mixture in a single tube, vial, well, or container.

All publications, patents, patent applications, and information available on the internet and mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions will be evident to those skilled in the art without departing from the scope of this disclosure. It should also be understood that various alternatives to the specific embodiments described herein are also within the scope of this disclosure.

The compositions, methods, and kits described herein generally describe detecting a target nucleic acid (e.g., a viral nucleic acid) in a biological sample. The methods, compositions, and kits disclosed herein generally involve lysis of the biological sample with a non-ionic detergent, reverse transcription, primer binding, amplification, and detection in a single reaction container. The container may be a test tube, a well of a microwell plate, a PCR tube, a microplate, or another vessel that is capable of holding the mixture as such mixture is otherwise described herein. In some embodiments, the reagents to create the mixture necessary for the aforementioned method are added at the same time along with a non-ionic detergent at an appropriate temperature to maximize reverse transcriptase activity, although in other embodiments, the reagents for the mixture may be added one-at-a-time to create the mixture. In some embodiments, the non-ionic detergent lyses the biological sample thus by-passing the need for traditional biological sample heat lysis (e.g., about 95° C.) and allowing the biological sample to be processed in a single container (e.g., tube). It will be understood that the disclosed methods and systems also allow for the by-passing of other forms of lysis of the biological sample/target nucleic acid.

As more fully described herein, amplification and detection includes real-time PCR or quantitative PCR (qPCR). In some embodiments, a sequence-specific detection method, such as a TaqMan Assay is used. In some embodiments, a dsDNA binding dye based detection, such as a SYBR dye (e.g., SYBR Green) is used.

Provided herein are methods for detecting a presence of a target nucleic acid in a biological sample, the method including creating a mixture in a container, the mixture including the biological sample, a non-ionic detergent, one or more primers for specifically binding to the target nucleic acid in the biological sample, or a complement thereof, one or more probes for the target nucleic acid, and, one or more polymerases, wherein the mixture is created prior to subjecting the target nucleic acid to a nucleic acid extraction or a lysis; incubating the mixture to react the non-ionic detergent with the biological sample; amplifying the target nucleic acid, or a complement thereof, by polymerization using the one or more polymerases to generate an amplified target nucleic acid product, or a complement thereof; and detecting the amplified target nucleic acid product or complement thereof with the one or more probes, thereby detecting the presence of the target nucleic acid in the biological sample.

In some embodiments, also provided herein are methods for detecting the presence of a viral nucleic acid in a biological sample, including creating a mixture in a container, the mixture including the biological sample, a non-ionic detergent, one or more primers for specifically binding to the viral nucleic acid in the biological sample, or a complement thereof, a nuclease inhibitor, one or more probes for the viral nucleic acid, and one or more polymerases, wherein the mixture is created prior to subjecting the viral nucleic acid to a nucleic acid extraction or lysis, incubating the mixture to react the non-ionic detergent with the biological sample, amplifying the viral nucleic acid, or a complement thereof, by polymerization using the one or more polymerases to generate an amplified viral nucleic acid product, or a complement thereof, and detecting the amplified nucleic acid product, or complement thereof, with the one or more probes, thereby detecting the presence of the viral nucleic acid in the biological sample.

In such embodiments, uracil DNA glycosylase (UDG) eliminates contaminating nucleic acids in the biological sample, and the nuclease inhibitors protect the target nucleic acid. In some embodiments, UDG is added to the reaction mixture before one or more (e.g., 2, 3, 4, 5, or more) reverse transcriptions reactions.

Also disclosed is a method for detecting the presence of a viral nucleic acid in a biological sample, the method including incubating a non-ionic detergent with the biological sample to react the non-ionic detergent with the biological sample; prior to subjecting the viral nucleic acid to either a nucleic acid extraction or lysis, contacting the biological sample and the non-ionic detergent with a mixture including one or more primers for specifically binding to the viral nucleic acid in the biological sample, or a complement thereof, one or more probes for the viral nucleic acid, and one or more polymerases, amplifying the viral nucleic acid, or a complement thereof, by polymerization using the one or more polymerase to generate an amplified viral nucleic acid product, or a complement thereof; and detecting the amplified viral nucleic acid product, or complement thereof, with the one or more probes, thereby detecting the presence of the virus in the biological sample.

Detergents can be ionic detergents or non-ionic detergents. In certain applications non-ionic detergents have beneficial properties. For example, non-ionic detergents, such as Tween®-20, NP-40, ECOSURF™ SA, and Triton® X-100, have the added benefit of not inhibiting PCR reactions. In contrast, even trace amounts as low as 0.01% of strong ionic detergents have been shown to inhibit PCR (Gelfand, D. H. and White, T. J., PCR Protocols: A Guide to Methods and Applications, Innis, M. A., Gelfand, D. H., Sninsky, J. J. and White, T. J., eds, Academic Press, San Diego, Calif., 129-41 (1990)).

In some embodiments, methods, compositions, or kits provided herein employ the non-ionic detergent Tween®-20, the non-ionic detergent Tween®-80, ECOSURF™ SA, and/or the non-ionic detergent NP-40. In some embodiments, methods, compositions, and kits provided herein employ the non-ionic detergent Triton® X-100 or a Brij detergent. For example, the Brij detergent can be Brij-58, although such example is provided for illustration and not limitation.

In some embodiments, non-ionic detergents are part of a mixture (e.g., more fully described in the Examples) including the reagents for target nucleic acid amplification and detection. Thus, in some embodiments, a pre-treatment comprising mixing the virus with the non-ionic detergent(s) for viral inactivation is not required.

In some embodiments, the non-ionic detergent (e.g., any of the non-ionic detergents described herein) is present at a concentration from about 0.001% to about 10.0%, at a concentration from about 1.0% (v/v) to about 5.0% (v/v), or at a concentration from about 0.005% (v/v) to about 9.5% (v/v), for example, from about 0.01% (v/v) to about 9.0% (v/v), from about 0.05 (v/v) percent to about 8.5% (v/v), from about 0.1% (v/v) to about 8.0% (v/v), from about 0.5% (v/v) to about 7.5% (v/v), from about 1.0% (v/v) to about 7.0% (v/v), from about 1.5% (v/v) to about 6.5% (v/v), from about 2.0% (v/v) to about 6.0% (v/v), from about 2.5% (v/v) to about 5.5% (v/v), from about 3.0% (v/v) to about 4.5% (v/v), or from about 3.5% (v/v) to about 4.0% (v/v).

In some embodiments, the non-ionic detergent (e.g., any of the non-ionic detergents described herein) is present at a concentration of at least about 0.001%, about 0.005% (v/v), about 0.01% (v/v), about 0.015% (v/v), about 0.02% (v/v), about 0.025% (v/v), about 0.03% (v/v), about 0.03 5% (v/v), about 0.04% (v/v), about 0.045% (v/v), about 0.05% (v/v), about 0.055% (v/v), about 0.06% (v/v), about 0.065% (v/v), about 0.07% (v/v), about 0.075% (v/v), about 0.08% (v/v), about 0.085% (v/v), about 0.09% (v/v), about 0.095% (v/v), about 0.1% (v/v), about 0.15% (v/v), about 0.2% (v/v), about 0.25% (v/v), about 0.3% (v/v), about 0.35% (v/v), about 0.4% (v/v), about 0.45% (v/v), about 0.5% (v/v), about 0.55% (v/v), about 0.6% (v/v), about 0.65% (v/v), about 0.7% (v/v), about 0.75% (v/v), about 0.8% (v/v), about 0.85% (v/v), about 0.9% (v/v), about 0.95% (v/v), about 1% (v/v), about 1.05% (v/v), about 0.25% (v/v), about 0.3% (v/v), about 0.3 5% (v/v), about 0.4% (v/v), about 0.45% (v/v), about 0.5% (v/v), about 0.5 5% (v/v), about 0.6% (v/v), about 0.65% (v/v), about 0.7% (v/v), about 0.75% (v/v), about 0.8% (v/v), about 0.85% (v/v), about 0.9% (v/v), about 0.95% (v/v), about 1% (v/v), about 1.05% (v/v), about 1.1% (v/v), about 1.15% (v/v), about 1.2% (v/v), about 1.25% (v/v), about 1.3% (v/v), about 1.3 5% (v/v), about 1.4% (v/v), about 1.45% (v/v), about 1.5% (v/v), about 2.4% (v/v), about 2.45% (v/v), about 2.5% (v/v), about 2.55% (v/v), about 2.6% (v/v), about 2.65% (v/v), about 2.7% (v/v), about 2.75% (v/v), about 2.8% (v/v), about 2.85% (v/v), about 2.9% (v/v), about 2.95% (v/v), about 3% (v/v), about 3.05% (v/v), about 3.1% (v/v), about 3.15% (v/v), about 3.2% (v/v), about 3.25% (v/v), about 3.3% (v/v), about 3.3 5% (v/v), about 3.4% (v/v), about 3.45% (v/v), about 3.5% (v/v), about 3.55% (v/v), about 3.6% (v/v), about 3.65% (v/v), about 3.7% (v/v), about 3.75% (v/v), about 3.8% (v/v), about 3.85% (v/v), about 3.9% (v/v), about 3.95% (v/v), about 4% (v/v), about 4.05% (v/v), about 4.1% (v/v), about 4.15% (v/v), about 4.2% (v/v), about 4.25% (v/v), about 4.3% (v/v), about 4.3 5% (v/v), about 4.4% (v/v), about 4.45% (v/v), about 4.5% (v/v), about 4.55% (v/v), about 4.6% (v/v), about 4.65% (v/v), about 4.7% (v/v), about 4.75% (v/v), about 4.8% (v/v), about 4.85% (v/v), about 4.9% (v/v), about 4.95% (v/v), about 5% (v/v), about 5.05% (v/v), about 5.1% (v/v), about 5.15% (v/v), about 5.2% (v/v), about 5.25% (v/v), about 5.3% (v/v), about 5.35% (v/v), about 5.4% (v/v), about 5.45% (v/v), about 5.5% (v/v), about 5.55% (v/v), about 5.6% (v/v), about 5.65% (v/v), about 5.7% (v/v), about 5.75% (v/v), about 5.8% (v/v), about 5.85% (v/v), about 5.9% (v/v), about 5.95% (v/v), about 6% (v/v), about 6.05% (v/v), about 6.1% (v/v), about 6.15% (v/v), about 6.2% (v/v), about 6.25% (v/v), about 6.3% (v/v), about 6.3 5% (v/v), about 6.4% (v/v), about 6.45% (v/v), about 6.5% (v/v), about 6.55% (v/v), about 6.6% (v/v), about 6.65% (v/v), about 6.7% (v/v), about 6.75% (v/v), about 6.8% (v/v), about 6.85% (v/v), about 6.9% (v/v), about 6.95% (v/v), about 7% (v/v), about 7.05% (v/v), about 7.1% (v/v), about 7.15% (v/v), about 7.2% (v/v), about 7.25% (v/v), about 7.3% (v/v), about 7.35% (v/v), about 7.4% (v/v), about 7.45% (v/v), about 7.5% (v/v), about 7.55% (v/v), about 7.6% (v/v), about 7.65% (v/v), about 7.7% (v/v), about 7.75% (v/v), about 7.8% (v/v), about 7.85% (v/v), about 7.9% (v/v), about 7.95% (v/v), about 8% (v/v), about 8.05% (v/v), about 8.1% (v/v), about 8.15% (v/v), about 8.2% (v/v), about 8.25% (v/v), about 8.3% (v/v), about 8.35% (v/v), about 8.4% (v/v), about 8.45% (v/v), about 8.5% (v/v), about 8.55% (v/v), about 8.6% (v/v), about 8.65% (v/v), about 8.7% (v/v), about 8.75% (v/v), about 8.8% (v/v), about 8.85% (v/v), about 8.9% (v/v), about 8.95% (v/v), about 9% (v/v), about 9.05% (v/v), about 9.1% (v/v), about 9.15% (v/v), about 9.2% (v/v), about 9.25% (v/v), about 9.3% (v/v), about 9.35% (v/v), about 9.4% (v/v), about 9.45% (v/v), about 9.5% (v/v), about 9.55% (v/v), about 9.6% (v/v), about 9.65% (v/v), about 9.7% (v/v), about 9.75% (v/v), about 9.8% (v/v), about 9.85% (v/v), about 9.9% (v/v), about 9.95% (v/v), or about 10% (v/v).

In some embodiments of methods provided herein, the non-ionic detergent is Tween®-20 present at a concentration of about 0.05% (v/v).

In some embodiments, the methods, compositions, and kits described herein lyse the biological sample without subjecting the target nucleic acid within the biological sample to a lysis or a nucleic acid extraction. For example, in some embodiments, the biological sample is lysed using heat at a temperature that is from about 35° C. to about 75° C., from about 37° C. to about 73° C., from about 39° C. to about 71° C., from about 41° C. to about 69° C., from about 43° C. to about 67° C., from about 45° to about 65, from about 47° C. to about 63° C., from about 49° C. to about 61° C., from about 51° C. to about 59° C., or from about 53° C. to about 57° C.

In some embodiments, the biological sample is lysed at a temperature at about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., or about 75° C.

In some examples of the disclosed methods, systems, and compositions, the biological sample is lysed for a period of time from about 0.5 minutes to about 25 minutes, from about 1 minute to about 24 minutes, from about 3 minutes to about 22 minutes, from about 5 minutes to about 20 minutes, from about 7 minutes to about 18 minutes, from about 9 minutes to about 16 minutes, from about 11 minutes to about 14 minutes, or from about 12 minutes to about 13 minutes.

The biological sample may be lysed for a period of time of about 1 minute to about 25 minutes, including, for example, about 1 minute, about 1.5 minutes, about 2 minutes, about 2.5 minutes, about 3 minutes, about 3.5 minutes, about 4 minutes, about 4.5 minutes, about 5 minutes, about 5.5 minutes, about 6 minutes, about 6.5 minutes, about 7 minutes, about 7.5 minutes, about 8 minutes, about 8.5 minutes, about 9 minutes, about 9.5 minutes, about 10 minutes, about 10.5 minutes, about 11 minutes, about 11.5 minutes, about 12 minutes, about 12.5 minutes, about 13 minutes, about 13.5 minutes, about 14 minutes, about 14.5 minutes, about 15 minutes, about 15.5 minutes, about 16 minutes, about 16.5 minutes, about 17 minutes, about 17.5 minutes, about 18 minutes, about 18.5 minutes, about 19 minutes, about 19.5 minutes, about 20 minutes, about 20.5 minutes, about 21 minutes, about 21.5 minutes, about 22 minutes, about 22.5 minutes, about 23 minutes, about 23.5 minutes, about 24 minutes, about 24.5 minutes, or about 25 minutes.

In some embodiments, the target nucleic acid that is detected is RNA (e.g., RNA from an RNA virus). To detect the presence of the target nucleic acid (e.g., RNA) reverse transcription can be performed, for example, with a polymerase, such as a reverse transcriptase. In some embodiments, more than one (e.g., 2, 3, 4, 5, or more) reverse transcription reactions are performed.

The reverse transcriptase may be a wild-type reverse transcriptase, or a derivative of a wild-type reverse transcriptase (e.g., a reverse transcriptase with an engineered mutation). For example, some derivatives of wild-type reverse transcriptases can have improved properties, such as for example, improved thermostability.

Non-limiting examples of reverse transcriptases that can be used in the methods and kits described herein include such reverse transcriptases as, MMLV, MMLV (RNase H minus), SuperScript II (Thermofisher), SuperScript III (Thermofisher), SuperScript IV (Thermofisher), RevertAid H Minus(Thermofisher), Maxima H (Thermofisher), ProtoScript II (NEB), EnzScript™ (Enzymatics), ABscript II (ABclonal), EpiScript™ RNase H—(Lucigen), or RocketScript (Bioneer).

In some embodiments, the reverse transcription reaction is performed at a temperature from about 35° C. to about 75° C., from about 37° C. to about 73° C., from about 39° C. to about 71° C., from about 41° C. to about 69° C., from about 43° C. to about 67° C., from about 45° C. to about 65° C., from about 47° C. to about 63° C., from about 49° C. to about 61° C., from about 51° C. to about 59° C., or from about 53° C. to about 57° C.

In some embodiments, the reverse transcription reaction is performed at a temperature of about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., or about 75° C.

In some embodiments, the reverse transcription reaction is performed for a period of time ranging from about 0.5 minutes to about 20 minutes, from about 2 minutes to 19 minutes, from about 3 minutes to about 18 minutes, from about 4 minutes to about 17 minutes, from about 5 minutes to about 16 minutes, from about 6 minutes to about 15 minutes, from about 7 minutes to about 14 minutes, from about 8 minutes to about 13 minutes, from about 9 minutes to about 12 minutes, or from about 10 to about 11 minutes.

In some embodiments, the reverse transcription reaction is performed for a period of time ranging about 0.5 minutes, about 1 minute, about 1.5 minutes, about 2 minutes, about 2.5 minutes, about 3 minutes, about 3.5 minutes, about 4 minutes, about 4.5 minutes, about 5 minutes, about 5.5 minutes, about 6 minutes, about 6.5 minutes, about 7 minutes, about 7.5 minutes, about 8 minutes, about 8.5 minutes, about 9 minutes, about 9.5 minutes, about 10 minutes, about 10.5 minutes, about 11 minutes, about 11.5 minutes, about 12 minutes, about 12.5 minutes, about 13 minutes, about 13.5 minutes, about 14 minutes, about 14.5 minutes, about 15 minutes, about 15.5 minutes, about 16 minutes, about 16.5 minutes, about 17 minutes, about 17.5 minutes, about 18 minutes, about 18.5 minutes, about 19 minutes, about 19.5 minutes, or about 20 minutes.

In some embodiments, nuclease inhibitor(s) are also included in the mixture. In some embodiments, the nuclease inhibitor(s) comprises an RNase inhibitor. For example, the RNase inhibitor can be included when detecting viral RNA (e.g., SARS-CoV-2) in a biological sample. In some embodiments, the nuclease inhibitor(s) comprises a DNase inhibitor. For example, the DNase inhibitor can be included in the mixture when detecting viral DNA.

In some embodiments, the RNase inhibitor(s) is a wild-type RNase inhibitor. In some embodiments, the RNase inhibitor(s) is a derivative of a wild-type RNase inhibitor (e.g., an RNase inhibitor with a mutation(s)). For example, the derivative RNase inhibitor can have improved performance, such as for example, improved thermostability. Some non-limiting examples of RNase inhibitors include: RNase Inhibitor (Takara), SUPERase⋅In™ RNase Inhibitor (Thermofisher), RNasin® Plus RNase Inhibitor (Promega), RNasin® RNase Inhibitor (Promega), or RNase Inhibitor (NEB).

In some embodiments, the DNase inhibitor is a wild type DNase inhibitor. In some embodiments, the DNase inhibitor is a derivative of a wild-type DNase inhibitor (e.g., a DNase inhibitor with a mutation(s)). For example, the derivative DNase inhibitor can have improved properties, such as for example, improved thermostability.

Uracil-DNA glycosylase (UDG) (also known as uracil-N glycosylase (UNG)) is an enzyme that can prevent mutagenesis by eliminating uracil from DNA molecules by cleaving the N-glycosidic bond and initiating the base-excision repair pathway. In some embodiments, UDG enzymes can be added to the mixture (e.g., the enzyme mix) to prevent contamination of uracils into synthesized DNA. In some embodiments the UDG is a wild-type UDG enzyme. In some embodiments, the UDG enzyme is a derivative of a wild-type UDG enzyme (e.g., a UDG enzyme with a mutation(s)). For example, a derivative of wild-type UDG enzyme can have improved performance, such as for example, improved thermostability. Some non-limiting examples of UDG enzymes include: Uracil-DNA Glycosylase (NEB, Thermofisher), Antarctic Thermolabile UDG (NEB), and Uracil-DNA Glycosylase, heat-labile (Roche, Thermofisher).

In example embodiments, amplifying viral nucleic acid in a biological sample aids in detecting the presence of a viral nucleic acid in the biological sample. In the case of an RNA virus, after reverse transcription is performed (e.g., post-lysis), amplification can be performed with a DNA polymerase. In the case of a DNA virus, amplification can be performed directly on the DNA present in the lysed biological sample.

Various amplification techniques and reagents are known to a person of ordinary skill in the art and are within the scope of the methods described herein. In some embodiments, a wild-type DNA polymerase, or a derivative of a wild type DNA polymerase, is used to amplify the target nucleic acid in the biological sample. The DNA polymerase can be a derivative of a wild-type DNA polymerase (e.g., a DNA polymerase with a mutation(s)). For example, the derivative of the DNA polymerase can have improved performance, such as for example, improved fidelity or improved thermostability (e.g., compatible with hot-start technology). Some non-limiting examples of suitable DNA polymerases include: Platinum Taq (Thermofisher), AmpliTaq™ (Thermofisher), Kapa Taq (Roche), MyTaq™ (Meridian), Immolase™ (Meridian), OneTaq (NEB), and Phoenix Taq (Enzymatics).

In molecular diagnostics, amplifying a target nucleic acid (e.g., a viral nucleic acid) in a biological sample can aid in detection of the target nucleic acid. In the disclosed methods, compositions, and kits, in the case of an RNA virus, after DNA is generated (e.g., by reverse transcription) the DNA can be amplified directly in a single reaction tube. For example, the DNA (e.g., DNA from the target nucleic acid) is amplified in the single reaction tube by isothermal nucleic acid amplification.

Isothermal amplification enables rapid and specific amplification of DNA at a constant temperature or range of temperatures, thus avoiding the requirement of thermal cycling used in traditional PCR. In contrast to the polymerase chain reaction (PCR) technology, in which the reaction is carried out with a series of alternating temperature cycles with the use of a thermal cycler (or thermocycler), isothermal amplification is carried out at a constant temperature, and does not require a thermal cycler. Thus, isothermal nucleic acid amplification can be used as an alternative to standard PCR reactions (e.g., a PCR reaction that requires heating to about 95° C. to denature double stranded DNA). Isothermal nucleic acid amplification generally does not require the use of a thermocycler, however, in some embodiments, isothermal amplification can be performed in a thermocycler. In some embodiments, isothermal amplification is faster than a standard PCR reaction. In some embodiments, isothermal amplification is a linear amplification (e.g., asymmetrical with a single primer), while in embodiments, isothermal amplification is an exponential amplification (e.g., with two primers).

Isothermal amplification can be performed at a temperature from about 35° C. to about 75° C. For example, isothermal amplification can be performed at a temperature from about 40° C. to about 70° C., from about 45° C. to about 65° C., from about 50° C. to about 60° C., or about 55° C.

Accordingly, isothermal amplification can be performed at a temperature of about 35° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., or about 75° C.

Isothermal amplification can be performed for a period of time from about 15 seconds to about 3 minutes, from about 30 seconds to about 2.5 minutes, from about 45 seconds to about 2 minutes, from about 1 minute to about 1.75 minutes, or from about 1.25 minutes to about 1.5 minutes.

It can thus be understood that in some examples, isothermal amplification can be performed for a period of time of about 15 seconds, about 16 seconds, about 17 seconds, about 18 seconds, about 19 seconds, about 20 seconds, about 21 seconds, about 22 seconds, about 23 seconds, about 24 seconds, about 25 seconds, about 26 seconds, about 27 seconds, about 28 seconds, about 29 seconds, about 30 seconds, about 31 seconds, about 32 seconds, about 33 seconds, about 34 seconds, about 35 seconds, about 36 seconds, about 37 seconds, about 38 seconds, about 39 seconds, about 40 seconds, about 41 seconds, about 42 seconds, about 43 seconds, about 44 seconds, about 45 seconds, about 46 seconds, about 47 seconds, about 48 seconds, about 49 seconds, about 50 seconds, about 51 seconds, about 52 seconds, about 53 seconds, about 54 seconds, about 55 seconds, about 56 seconds, about 57 seconds, about 58 seconds, about 59 seconds, about 60 seconds, about 61 seconds, about 62 seconds, about 63 seconds, about 64 seconds, about 65 seconds, about 66 seconds, about 67 seconds, about 68 seconds, about 69 seconds, about 70 seconds, about 71 seconds, about 72 seconds, about 73 seconds, about 74 seconds, about 75 seconds, about 76 seconds, about 77 seconds, about 78 seconds, about 79 seconds, about 80 seconds, about 81 seconds, about 82 seconds, about 83 seconds, about 84 seconds, about 85 seconds, about 86 seconds, about 87 seconds, about 88 seconds, about 89 seconds, about 90 seconds, about 91 seconds, about 92 seconds, about 93 seconds, about 94 seconds, about 95 seconds, about 96 seconds, about 97 seconds, about 98 seconds, about 99 seconds, about 100 seconds, about 101 seconds, about 102 seconds, about 103 seconds, about 104 seconds, about 105 seconds, about 106 seconds, about 107 seconds, about 108 seconds, about 109 seconds, about 110 seconds, about 111 seconds, about 112 seconds, about 113 seconds, about 114 seconds, about 115 seconds, about 116 seconds, about 117 seconds, about 118 seconds, about 119 seconds, about 120 seconds, about 121 seconds, about 122 seconds, about 123 seconds, about 124 seconds, about 125 seconds, about 126 seconds, about 127 seconds, about 128 seconds, about 129 seconds, about 130 seconds, about 131 seconds, about 132 seconds, about 133 seconds, about 134 seconds, about 135 seconds, about 136 seconds, about 137 seconds, about 138 seconds, about 139 seconds, about 140 seconds, about 141 seconds, about 142 seconds, about 143 seconds, about 144 seconds, about 145 seconds, about 146 seconds, about 147 seconds, about 148 seconds, about 149 seconds, about 150 seconds, about 151 seconds, about 152 seconds, about 153 seconds, about 154 seconds, about 155 seconds, about 156 seconds, about 157 seconds, about 158 seconds, about 159 seconds, about 160 seconds, about 161 seconds, about 162 seconds, about 163 seconds, about 164 seconds, about 165 seconds, about 166 seconds, about 167 seconds, about 168 seconds, about 169 seconds, about 170 seconds, about 171 seconds, about 172 seconds, about 173 seconds, about 174 seconds, about 175 seconds, about 176 seconds, about 177 seconds, about 178 seconds, about 179 seconds, or about 180 seconds.

Non-limiting examples of suitable isothermal nucleic acid amplification techniques include, rolling circle amplification, loop-mediated isothermal amplification of DNA (LAMP), recombinase polymerase amplification, and helicase-dependent amplification (See e.g., Gill and Ghaemi, Nucleic acid isothermal amplification technologies: a review, Nucleosides, Nucleotides, & Nucleic Acids, 27(3), 224-43, doi: 10.1080/15257770701845204 (2008)).

In embodiments, the isothermal nucleic acid amplification comprises a helicase-dependent nucleic acid amplification. Strands of double stranded DNA are first separated by a DNA helicase and coated by single-stranded DNA (ssDNA)-binding proteins. Thereafter, two sequence specific primers hybridize to each border of the DNA template. DNA polymerases are then used to extend the primers annealed to the templates to produce a double stranded DNA and the two newly synthesized DNA products are then used as substrates by DNA helicases, entering the next round of the reaction. Thus, a simultaneous chain reaction develops, resulting in exponential amplification of the selected target sequence (See e.g., Vincent, et. al., Helicase-dependent isothermal DNA amplification, EMBO Rep., 795-800 (2004)).

In embodiments, the isothermal nucleic acid amplification comprises a recombinase polymerase nucleic acid amplification (See e.g., Piepenburg, et al., DNA Detection Using Recombinant Proteins, PLoS Biol., 4, 7 e204 (2006) and Li, et. al., Review: a comprehensive summary of a decade development of the recombinase polymerase amplification, Analyst, 144, 31-67, doi: 10.1039/C8AN01621F (2019)). Recombinase polymerase amplification (RPA) can amplify DNA, however, adding a reverse transcriptase enzyme to an RPA reaction can detect RNA as well as DNA. In some embodiments, the isothermal amplification is an RPA reaction with a reverse transcriptase.

In embodiments, the isothermal nucleic acid amplification comprises a loop-mediated isothermal amplification (LAMP). LAMP is a single-tube technique for the amplification of DNA and used in molecular diagnostic applications. Reverse Transcription Loop-mediated Isothermal Amplification (RT-LAMP) combines LAMP with reverse transcription to allow the detection of RNA (e.g., RNA from an RNA virus).

In embodiments, the isothermal nucleic acid amplification comprises rolling circle amplification. Rolling circle amplification (RCA) is a process of unidirectional nucleic acid replication that can rapidly synthesize multiple copies of DNA or RNA, for example, circular nucleic acids such as plasmids, bacteriophage genomes, and circular RNA genomes of viroids. Some eukaryotic viruses also replicate their DNA or RNA via the rolling circle mechanism. Additionally, RCA can be used as an isothermal DNA amplification technique, typically in molecular biology applications as a method of signal amplification.

Generally, isothermal amplification techniques use standard PCR reagents (e.g., buffer, dNTPs etc.) known to a person of ordinary skill in the art. Some isothermal amplification techniques can require additional reagents. For example, helicase dependent nucleic acid amplification uses a single-strand binding protein and an accessory protein. In another example, recombinase polymerase nucleic acid amplification uses recombinase (e.g., T4 UvsX), recombinase loading factor (e.g., TF UvsY), single-strand binding protein (e.g., T4 gp32), crowding agent (e.g., PEG-35K), and ATP. For example, in LAMP, the target sequence is amplified at a constant temperature using either two or three sets of primers, although additional sets of primers can be used, and a polymerase.

The methods described here can be applied, but not limited to, one-step real-time RT-PCR, or one-step isothermal amplification.

In some embodiments, quantitatively measuring the presence of a target nucleic acid (e.g., a viral nucleic acid) or a complement thereof, in a biological sample, includes using quantitative PCR. Methods of qPCR are well-known to a person of ordinary skill in the art. In some embodiments, qPCR includes a “TAQMAN™” assay. For example, TAQMAN probes can be designed to detect one or more target nucleic acids in a biological sample, including a control target nucleic acid. In some embodiments, qPCR includes the use of a dye, and for example, the dye may be a DNA intercalating dye such as “SYBR®” dye (e.g., SYBR Green). In embodiments, the quantification of genetic material is determined by optical absorbance in conjunction with real-time PCR.

Provided herein are methods of detecting a virus in a biological sample. In some embodiments, the virus is a SARS-CoV-2 virus. In some embodiments, detecting the SARS-CoV-2 virus includes detecting a gene of the SARS-CoV-2 virus in the biological sample. In some embodiments, the SARS-CoV-2 gene is the SARS-CoV-2 open reading frame (ORF) gene. In some embodiments, the SARS-CoV-2 gene is the SARS-CoV-2 N gene. In some embodiments, the biological sample is obtained from a human.

Thus, provided herein are methods of diagnosing a human with COVID-19 disease where detecting the SARS-CoV-2 virus in a biological sample (e.g., obtained from a human biological sample) includes detecting COVID-19 disease.

Kits

Also provided herein are kits including i) one or more polymerases, ii) one or more primers for a viral nucleic acid; iii) a non-ionic detergent, (iv) one or more probes; and (v) instructions for creating a mixture including a biological sample containing the viral nucleic acid, the one or more polymerases, the one or primers, the non-ionic detergent, and the one or more probes, wherein the mixture is created prior to subjecting the biological sample (or the target nucleic acid) to a nucleic acid extraction or a lysis.

In some kits, the one or more polymerases include one or more isothermal polymerases. In some kits, the polymerase is for a helicase-dependent amplification reaction, a recombinase polymerase amplification reaction, a loop-mediated isothermal amplification reaction, and/or a rolling circle amplification reaction.

In some kits, the one or more polymerases includes a reverse transcriptase. Some non-limiting examples that can be used in the kits described herein include such reverse transcriptases as, MMLV, MMLV (RNase H minus), SuperScript II (Thermofisher), SuperScript III (Thermofisher), SuperScript IV (Thermofisher), RevertAid H Minus (Thermofisher), Maxima H (Thermofisher), ProtoScript II (NEB), EnzScript™ (Enzymatics), ABscript II (ABclonal), EpiScript™ RNase H—(Lucigen), or RocketScript (Bioneer).

In some kits, the non-ionic detergent is selected from a group consisting of Tween® 20, Tween® 80, Triton X-100, NP 40, ECOSURF™ SA, Brij-58, and combinations thereof.

In some kits, the kit includes one or more RNase inhibitors. Non-limiting examples of the one or more RNase inhibitors that can be included in a kit include: RNase Inhibitor (Takara), SUPERase⋅In™ RNase Inhibitor (Thermofisher), RNasin® Plus RNase Inhibitor (Promega), RNasin® RNase Inhibitor (Promega), or RNase Inhibitor (NEB), and combinations thereof.

In some kits, one or more primers specifically bind (e.g., hybridize) to an RNA viral nucleic acid, such as, for example, the SARS-CoV-2 viral nucleic acid, the SARS-CoV-2 N gene, the SARS-CoV-2 ORF gene, and/or an MS2 bacteriophage.

In some kits, one or more primers specifically bind (e.g., hybridize) to a DNA viral nucleic acid.

In some kits, the kit includes instructions for detecting the presence of a virus in a biological sample.

In some kits, the kit includes fluorescent nucleotides for a quantitative PCR reaction. In some kits, the kit includes a non-sequence specific double-stranded dye.

In some kits, the kit includes a uracil-DNA glycosylase. Some non-limiting examples of uracil-DNA glycosylases include: Uracil-DNA Glycosylase (NEB, Thermofisher), Antarctic Thermolabile UDG (NEB), and Uracil-DNA Glycosylase, heat-labile (Roche, Thermofisher).

### EXAMPLES

### Example 1

### Detergent Improves Lysis Efficiency

In a real-time PCR assay a positive reaction was detected by accumulation of a fluorescent signal. The Ct (cycle threshold) is defined as the number of cycles required for the fluorescent signal to cross the background threshold, thus detecting a target nucleic acid (e.g., a viral nucleic acid) in a biological sample.

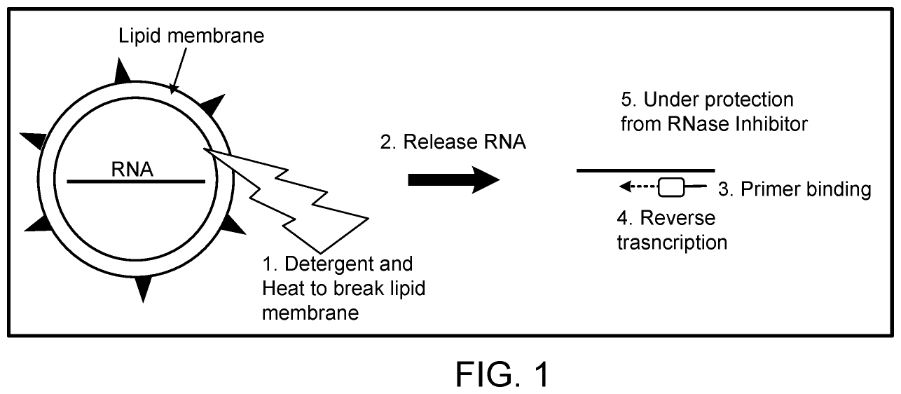

FIG. 1 shows an exemplary viral extraction and amplification scheme from a biological sample. FIG. 1 shows lysis with a detergent and/or heat to disrupt the lipid bi-layer of the virus. The viral nucleic acid (e.g., RNA) is released and sequence specific primers and a reverse transcriptase generates a complement of the viral nucleic acid. Additional enzymes, such as RNase inhibitors and additional polymerases (e.g., DNA polymerases) can also be present. While the exemplary viral extraction scheme shown in FIG. 1 describes RNA extraction and amplification, the methods described herein can be adapted for detection of DNA viral nucleic acid as well.

Gamma-irradiated SARS-Related Coronavirus 2 (SARS-CoV-2) was obtained from BEI resources (NR-52287 SARS-Related Coronavirus 2, Isolate USA-WA1/2020) and detection of the virus was performed with the PerkinElmer™ New Coronavirus Nucleic Acid Detection Kit (PerkinElmer, Waltham, Mass., USA).

A synthetic SARS-CoV-2 RNA construct (Control 2 MN908947.3 Wuhan-Hu-1, SKU: 102024) from Twist Bioscience was used as a positive control. No RNA extraction or lysis from a sample of synthetic RNA construct was necessary. Experiments were performed on a QuantStudio™ Dx 96-well real-time PCR instrument (Thermo Fisher, Waltham, Mass., USA).

25 μL of master mix (30 μL total for each PCR reaction) was prepared according to the following formulation:

7.5 μL nCoV reagent A (MgCl₂, Tris-HCl, dNTP mix (including dUTP) nCoV reagent A)

1.5 μL nCoV reagent B (oligonucleotide mix, including primers and probes)

1 μL nCoV enzyme mix (including heat-labile UDG, hot-start Taq DNA polymerase, reverse transcriptase, and RNase inhibitor)

5 μL nCoV internal control (Bacteriophage MS2 RNA),

10 μL detergent (Tween®-20, final concentration 0.05% in reaction) or water.

5 μL of sample: synthetic SARS-CoV-2 RNA construct (Twist Bioscience) 1000 cp per reaction (cp=copy number), or SARS-CoV-2 virus (1000 cp per rxn) with or without heat treatment (95° C. for 10 min) was added to the master mix. Each condition was tested in triplicate and shown below in Table 1. The thermocycler program is shown in Table 2. Reverse transcription was performed during thermocycler steps 2, 3, and 4 (e.g., at 55° C., 60° C., and 65° C., respectively). Step 1 in Table 2 (37° C. for 2 minutes) is the desired temperature for heat-labile UDG, to degrade carry-over PCR contamination.

TABLE 1

| | | | | Require |
|---|---|---|---|---|
| | | 95° C. | | lysis for |
| | | for 10 | | amplification |
| Condition | Sample Type | min | Detergent | and detection? |
| 1 | SARS-CoV-2 virus | + | − | Yes |
| 2 | SARS-CoV-2 virus | − | − | Yes |
| 3 | SARS-CoV-2 virus | − | + | Yes |
| 4 | Synthetic RNA Construct | − | − | No |
| 5 | Synthetic RNA Construct | − | + | No |

TABLE 2

| | | | Number of |
|---|---|---|---|
| Step | Temperature | Time | Cycles |
| 1 | 37° C. | 2 minutes | 1 |
| 2 | 55° C. | 5 minutes | 1 |
| 3 | 60° C. | 5 minutes | 1 |
| 4 | 65° C. | 5 minutes | 1 |
| 5 | 94° C. | 10 minutes | |
| 6 | 94° C. | 10 seconds | 45 |
| | 55° C. | 15 seconds | |
| | 65° C.* | 45 seconds | |

*Represents fluorescence collection

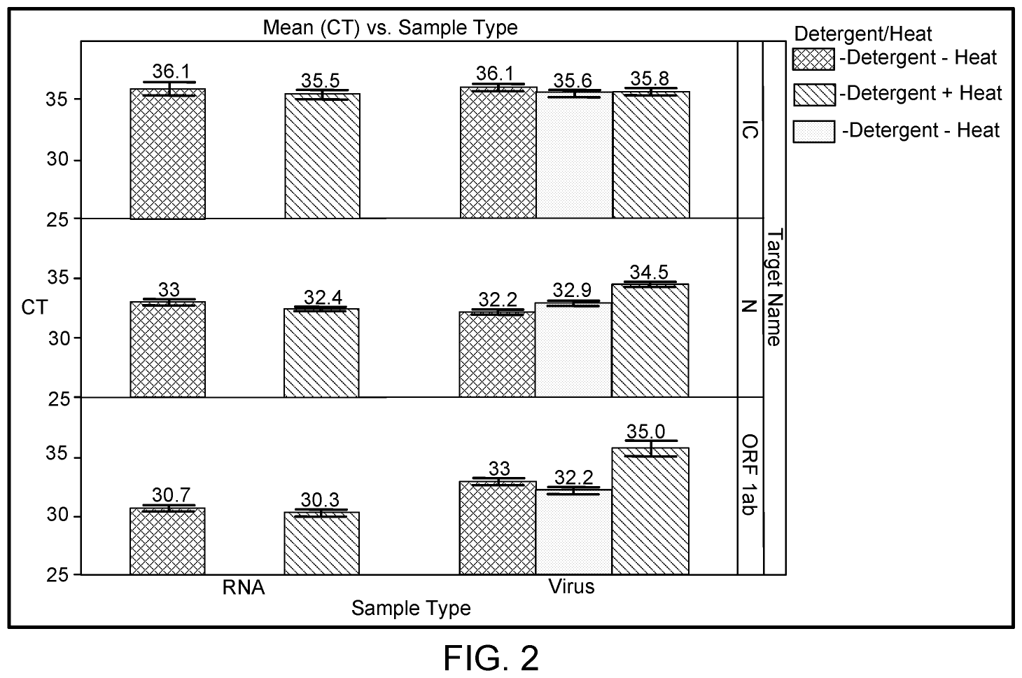

FIG. 2 shows a chart indicating the mean Ct threshold of the N, ORF1ab, and IC genes. The “N” and “ORF1ab” genes are known, target genes from SARS-CoV-2. IC is an internal control gene from bacteriophage MS2. The experiment was performed on both the synthetic RNA construct (RNA) and the SARS-CoV-2 virus (Virus) under combinations of heat and/or detergent. The data demonstrate that the presence of the detergent (e.g., Tween®-20) did not interfere with the amplification and detection for purified RNA from synthetic RNA construct. For example, similar Cts were achieved for each gene tested (IC gene: 36.1 Ct vs. 35.5; N gene: 33 vs. 32.4, ORF1ab gene: 30.7 vs. 30.3; detergent vs. no detergent, respectively) in the presence of the detergent.

The data also demonstrate that the detergent improved the lysis efficiency without performing target nucleic acid/biological sample lysis, e.g., heat lysis (e.g., heating at 95° C. for 10 min) or nucleic acid extraction. When the SARS-CoV-2 samples were treated with detergent only, the Cts of N (32.2 vs. 34.5) and ORF1ab (33 vs. 35.8) were reduced by about 2-3 Cts. Lysing the SARS-CoV-2 virus with a detergent achieved about a 4-10 fold improvement in lysis efficiency and achieves lysis efficiency similar to lysis via heat.

### EXAMPLE 2

### Detergent Improves Detection Sensitivity

To demonstrate that detergent can improve overall detection sensitive, the experiment was designed to detect 20 cp of inactivated virus with or without detergent. Similar experiment settings were performed, with the thermocycler program listed in Table 3, which has lower temperature (50° C.) for reverse transcription (thermocycler step 2). Step 1 in Table 3 (37° C. for 2 minutes) is the desired temperature for heat-labile UDG, to degrade carry-over PCR contamination. Step 1 is not part of virus lysis or reverse transcription.

TABLE 3

| | | | Number of |
|---|---|---|---|
| Step | Temperature | Time | Cycles |
| 1 | 37° C. | 2 minutes | 1 |
| 2 | 50° C. | 15 minutes | 1 |
| 3 | 94° C. | 10 minutes | 1 |
| 4 | 94° C. | 10 seconds | 45 |
| | 55° C. | 15 seconds | |
| | 65° C. | 45 seconds | |

*Represents fluorescence collection

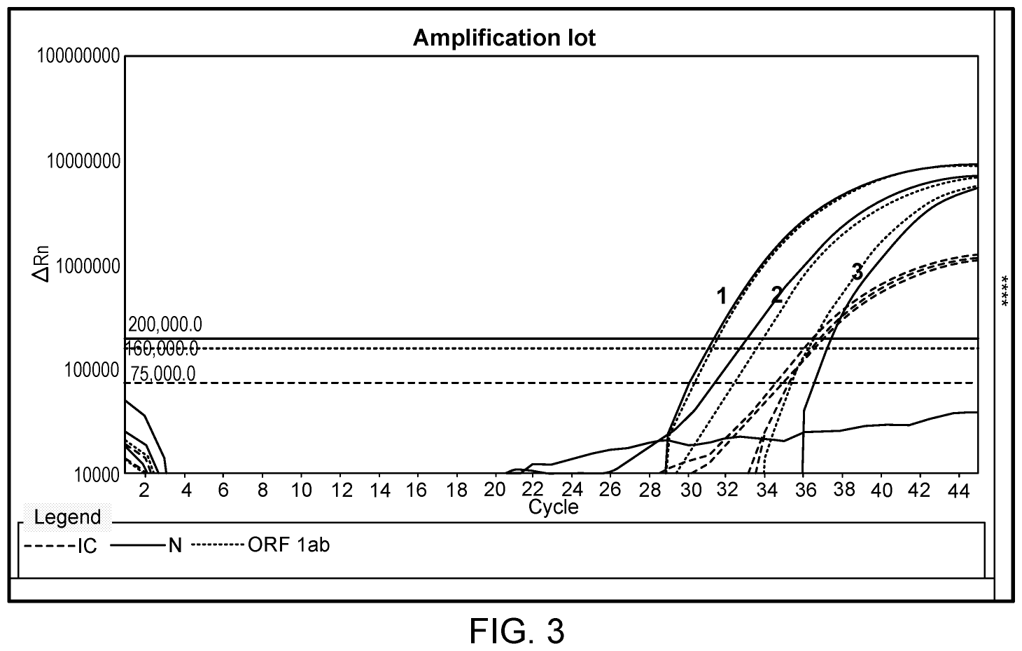

FIG. 3 is a graph showing an amplification plot. The numbers (e.g., 1, 2, 3, and 4) correspond to the following conditions:

1: 1,000 cp SARS-CoV-2 virus with detergent

2: 1,000 cp SARS-CoV-2 virus without detergent

3: 20 cp SARS-CoV-2 virus with detergent

4: 20 cp SARS-CoV-2 virus without detergent

For conditions 1-3, the N gene and the ORF1ab gene from the SARS-CoV-2 virus were detected. The solid lines represents the N gene and the short-dashed lines represent the ORF1ab gene. Table 4 summarizes the Ct mean and standard deviation (N=3). The overall result summary is listed in Table 4 for the both the SARS-CoV-2 samples (Virus) and the internal control (IC) samples (RNA).

TABLE 4

| | | | | IC | | | N | | | ORFlab | | |
|---|---|---|---|---|---|---|---|---|---|---|---|---|
| | | Input | | | CT | | | CT | | | CT | |
| | Sample | conc. | | CT | Std | | | Std | | | Std | |
| Condition | type | (cp/rxn) | Detergent | Mean | Dev | N | CT Mean | Dev | N | CT Mean | Dev | N |
| 1 | Virus | 1000 | Yes | 35.22 | 0.15 | 3 | 31.53 | 0.19 | 3 | 31.84 | 0.49 | 3 |
| 2 | Virus | 1000 | No | 35.38 | 0.58 | 3 | 33.04 | 0.21 | 3 | 33.92 | 0.33 | 3 |
| 3 | Virus | 20 | Yes | 35.37 | 0.47 | 3 | 38.12 | 0.93 | 3 | 37.25 | 1.31 | 3 |
| 4 | Virus | 20 | No | 34.64 | 0.18 | 3 | Undetermined | | 3 | Undetermined | | 3 |
| | RNA | 1000 | Yes | 35.05 | 0.31 | 3 | 32.47 | 0.22 | 3 | 30.23 | 0.15 | 3 |
| | RNA | 1000 | No | 34.78 | 0.69 | 3 | 32.11 | 0.34 | 3 | 30.15 | 0.23 | 3 |

The data in Table 4 demonstrate that the presence of a detergent did not interfere with amplification and detection for the synthetic RNA construct. The presence of the detergent improved detection sensitivity of the SARS-CoV-2 virus to 20 cp per rxn (e.g., compare row 3 and 4). Additionally, the improved lysis efficiency was also achieved at a lower temperature, 50° C., thus removing the need for performing lysis and thereby reducing the total reaction time.

### EXAMPLE 3

### Detergent Selection

To evaluate different detergents and the detergent concentrations in the final reaction, the sample protocol described in Example 1 was followed to detect 1,000 cp of inactivated SARS-CoV-2 virus with different detergents at different concentrations and with water (e.g., no detergent). Several detergents were tested, including NP 40, Tween® 20, and Triton™ X-100. All samples were processed according to the protocol described in Example 1, including the thermocycler program listed in Table 2 above. Table 5 summarizes the Ct Mean and standard deviation results for the IC (control), N, and ORF1ab genes (N=3) of the different detergents at different concentrations.

TABLE 5

| | IC | | | N | | | ORFlab | | |
|---|---|---|---|---|---|---|---|---|---|
| | | CT | | | CT | | | CT | |
| Sample (Detergent | | Std | | | Std | | | Std | |
| and concentration) | Ct Mean | Dev | N | Ct Mean | Dev | N | Ct Mean | Dev | N |
| NP 40 0.1% | 35.95 | 0.24 | 3 | 33.14 | 0.33 | 3 | 33.79 | 0.61 | 3 |
| Tween ^{®} 20 0.1% | 36.50 | 0.23 | 3 | 33.15 | 0.40 | 3 | 33.84 | 0.11 | 3 |
| Tween ^{®} 20 1% | 35.39 | 0.34 | 3 | 32.26 | 0.51 | 3 | 32.80 | 0.80 | 3 |
| Tween ^{®} 20 3.3% | 32.52 | 0.70 | 3 | 31.32 | 0.53 | 3 | 34.46 | 0.53 | 3 |
| Triton ™ X-100 0.1% | 36.45 | 0.22 | 3 | 33.84 | 0.23 | 3 | 34.36 | 0.51 | 3 |
| Triton ™ X-100 1% | 36.49 | 0.16 | 3 | 33.69 | 0.61 | 3 | 33.86 | 0.46 | 3 |
| Water | 35.69 | 0.62 | 3 | 34.43 | 0.50 | 3 | 36.25 | 1.20 | 3 |

Table 5 demonstrates that different non-ionic detergents improved SARS-CoV-2 viral detection efficiency based on Ct number relative to water (Ct mean=35.69) at different concentrations. For example, Tween® 20 at a final concentration of 3.3% reduced the Ct Mean from 35.69 (water) to 32.52, resulting in a more efficient and sensitive assay.

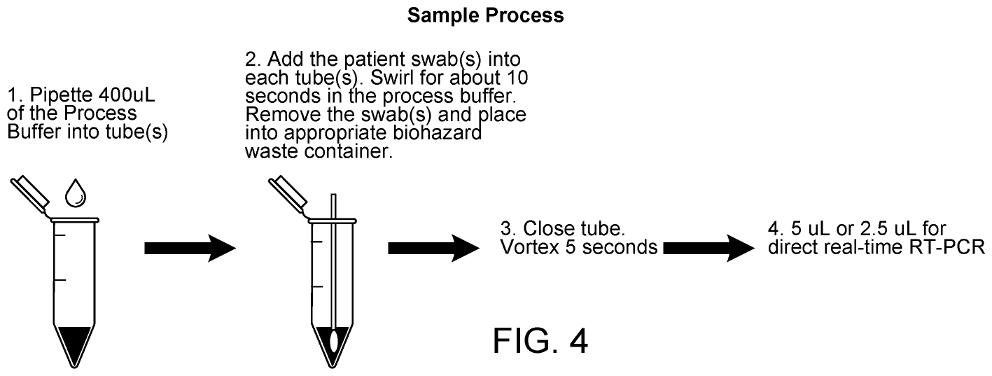

FIG. 4 demonstrates an example of a biological sample on a swab. In this example the swab is added to a tube with a volume of process buffer (without any lysis components) and agitated for a period of time in the solution. The swab is then removed and discarded and the solution in the tube is vortexed and a volume of the biological sample in solution is used in the methods described herein for the detection of a target nucleic acid.

Nasopharyngeal (NP) and oropharyngeal (OP) specimens should be collected and placed in a clean, dry collection tube. Specimen should be transported and tested as soon as possible after collection. The specimens are stable for up to 24 hours at room temperature or up to 72 hours when stored between 2° C. and 8° C. If the specimen cannot be tested within this time frame, they should be frozen at −70° C. or colder until testing can resume based on CDC guidelines. Avoid freezing and thawing specimens. Viability of some pathogens from specimens that are frozen and then thawed is greatly diminished and may result in false-negative test results.

### OTHER EMBODIMENTS

It is to be understood that while the methods, compositions, and kits have been described in conjunction with the detailed description thereof, the forgoing description is intended to illustrate and not limit the scope of the methods, compositions, and kits described herein, which is defined only by the scope of the appended claims. Other aspects, advantages, and modifications are thus understood to be and intended to be within the scope of the following claims.

What is claimed is:

1. A method for detecting a presence of a target nucleic acid in a biological sample, the method comprising:
creating a mixture in a container, the mixture comprising:
the biological sample,
a non-ionic detergent,
one or more primers for specifically binding to the target nucleic acid in the biological sample, or a complement thereof,
one or more probes for the target nucleic acid, and
one or more polymerases,
wherein the mixture is created prior to subjecting the target nucleic acid to a nucleic acid extraction or lysis;
incubating the mixture to react the non-ionic detergent with the biological sample;
amplifying the target nucleic acid, or a complement thereof, by polymerization using the one or more polymerases to generate an amplified target nucleic acid product, or a complement thereof; and
detecting the amplified target nucleic acid product or complement thereof with the one or more probes, thereby detecting the presence of the target nucleic acid in the biological sample.

2. The method of claim 1, wherein the non-ionic detergent is selected from a group consisting of: Tween 20, Tween 80, Triton X-100, NP 40, ECOSURF™ SA, Brij-58, and combinations thereof.

3. The method of claim 2, wherein the non-ionic detergent is present at a concentration from about 0.01% to about 10.0%.

4. The method of claim 1, wherein the biological sample is lysed for a period of time from about 30 seconds to about 20 minutes at a temperature from about 35° C. to about 75° C.

5. The method of claim 1, further comprising contacting the mixture with an RNase inhibitor and/or a uracil-DNA glycosylase.

6. The method of claim 1, wherein the target nucleic acid is a viral nucleic acid comprising DNA.

7. The method of claim 1, wherein the target nucleic acid is a viral nucleic acid comprising RNA.

8. The method of claim 7, wherein the viral nucleic acid is reverse transcribed using a reverse transcriptase selected from a group consisting of: MMLV, MMLV (RNase H minus), SuperScript II, SuperScript III, SuperScript IV, RevertAid H Minus, Maxima H, ProtoScript II, EnzScript™, ABscript II, EpiScript™, or RocketScript (Bioneer), and combinations thereof two or more times at a temperature of about 50° C. to about 70° C.

9. The method of claim 7, wherein the viral nucleic acid comprises viral nucleic acid from a bacteriophage, wherein the bacteriophage is an MS2 bacteriophage.

10. The method of claim 9, further comprising detecting a control nucleic acid, wherein the control nucleic acid is a MS2 bacteriophage gene.

11. The method of claim 7, wherein the viral nucleic acid comprises viral nucleic acid from a coronavirus and, optionally, wherein the coronavirus comprises a SARS-CoV-2 virus.

12. The method of claim 11, further comprising detecting the SARS-CoV-2 virus in the biological sample, wherein detecting the SARS-CoV-2 virus in the biological sample comprises detecting a SARS-CoV2 N gene and/or a SARS-CoV-2 ORF gene.

13. The method of claim 1, wherein the biological sample is obtained from a human, and optionally, wherein diagnosing the human with COVID-19 disease comprises detecting the SARS-CoV-2 virus in the human biological sample.

14. The method of claim 1, further comprising isothermally amplifying the target nucleic acid, wherein isothermally amplifying comprises one of a helicase-dependent amplification, a loop mediated isothermal amplification, a recombinase polymerase amplification, or a rolling circle amplification.

15. The method of claim 1, wherein the one or more polymerases comprises a DNA polymerase.

16. The method of claim 1, wherein the amplified target nucleic acid product is detected using a quantitative PCR method with the one or more probes.

17. The method of claim 16, wherein the quantitative PCR method comprises a real-time PCR assay and, optionally, wherein the quantitative PCR method comprises a TaqMan™ assay.

18. The method of claim 16, wherein the quantitative PCR method employs a non-sequence-specific double-stranded DNA-binding dye to detect the amplified target nucleic acid product and wherein the non-sequence specific double-stranded DNA binding dye is SYBR green.

19. A method for detecting the presence of a viral nucleic acid in a biological sample, the method comprising:
incubating a non-ionic detergent with the biological sample to react the non-ionic detergent with the biological sample;
prior to subjecting the viral nucleic acid to a nucleic acid extraction or lysis, contacting the biological sample and the non-ionic detergent with a mixture comprising:
one or more primers for specifically binding to the viral nucleic acid in the biological sample, or a complement thereof,
one or more probes for the viral nucleic acid, and
one or more polymerases;
amplifying the viral nucleic acid, or a complement thereof, by polymerization using the one or more polymerases to generate an amplified viral nucleic acid product, or a complement thereof; and
detecting the amplified viral nucleic acid product, or complement thereof, with the one or more probes, thereby detecting the presence of the virus in the biological sample.

20. A kit comprising:
(i) one or more polymerases;
(ii) one or more primers for a viral nucleic acid;
(iii) a non-ionic detergent;
(iv) one or more probes; and
(v) instructions for creating a mixture comprising a biological sample containing the viral nucleic acid, the one or more polymerases, the one or primers, the non-ionic detergent, and the one or more probes, wherein the mixture is created prior to subjecting the viral nucleic acid to a nucleic acid extraction or a lysis.
